(19) United States
(12) Patent Application Publication
(10) Pub. No.: US 2026/0090750 A1
(43) Pub. Date: Apr. 2, 2026

(54) SYSTEMS AND METHODS FOR AUTOMATICALLY ASSESSING A PHYSIOLOGICAL STATE

(71) Applicant: RightEye, LLC, Bethesda, MD (US)

(72) Inventors: Melissa HUNFALVAY, Bethesda, MD (US); Adam Todd GROSS, Bethesda, MD (US); Takumi BOLTE, Bethesda, MD (US)

(73) Assignee: RightEye, LLC, Bethesda, MD (US)

(21) Appl. No.: 19/343,212

(22) Filed: Sep. 29, 2025

Related U.S. Application Data
(60) Provisional application No. 63/701,771, filed on Oct. 1, 2024.

(51) Int. Cl. A61B 5/16; A61B 5/00

(52) U.S. Cl. CPC A61B 5/165; A61B 5/163; A61B 5/7264

(57) ABSTRACT

Automatically assessing a physiological state of a user may include capturing, by an eye tracking device, eye movement of a user as eye movement data, providing the eye movement data to a machine-learning model trained to identify associations between one or more patterns in the eye movement data and one or more characteristics of one or more physiological states, outputting, by the machine-learning model, the physiological state of the user based on the identified associations, determining an intervention for the user based on the output physiological state, and outputting the intervention.

### RELATED APPLICATION(S)

This application is a nonprovisional of and claims the benefit of priority to U.S. Provisional Application No. 63/701,771, filed Oct. 1, 2024, the entire disclosure of which is hereby incorporated herein by reference.

### TECHNICAL FIELD

Various embodiments of this disclosure relate generally to machine-learning-based and/or artificial intelligence-based techniques for determining physical and mental characteristics and conditions, and, more particularly, to systems and methods for automatically assessing reader comprehension, confusion, and other conditions, and providing interventions and/or corrections.

### BACKGROUND

Reading is a fundamental skill for interacting with and understanding the world. Conventionally, the extent to which a person may read and understand written material may only be assessed after the fact (e.g., by determining that written instructions were skipped, misunderstood, or that mistakes were made by the reader, by determining that the reader has a lack of retention or understanding of the written subject matter that they ostensibly read, and etc.). While many factors may affect whether and to what extent a reader comprehends or is confused by written material, determining the presence or effect of such factors on the reader is generally difficult or impossible to do while the user is reading (e.g., in real-time/simultaneously as the reader is reading). These and other circumstances may interfere with a learning process of the reader or may reduce the reader's preparedness or effectiveness. Moreover, by the time such factors or their effects become known, it may be too late to effectively intervene and/or make corrections to reading behaviors.

Unless otherwise indicated herein, the materials described in this section are not prior art to this application and are not admitted to be prior art, or suggestions of the prior art, by inclusion in this section.

### SUMMARY OF THE DISCLOSURE

According to certain aspects of the present disclosure, systems and methods are disclosed for automatically assessing a physiological state of a user. In one embodiment, a computer-implemented method is disclosed. The method may include capturing, by an eye tracking device, eye movement of a user as eye movement data. The method may further include providing the eye movement data to a machine-learning model trained to identify associations between one or more patterns in the eye movement data and one or more characteristics of one or more physiological states. The method may further include outputting, by the machine-learning model, the physiological state of the user based on the identified associations. The method may further include determining an intervention for the user based on the output physiological state. The method may further include outputting the intervention.

In accordance with another embodiment, a system for automatically assessing a physiological state of a user may include an eye tracking device configured to capture eye movement of the user, the eye tracking device in electronic communication with a user device, one or more processors, and a memory including instructions, which, when executed by the one or more processors, cause the system to perform operations. The operations may include capturing, by the eye tracking device in electronic communication with the user device, the eye movement as eye movement data. The operations may further include providing the eye movement data to a machine-learning model trained to identify associations between one or more patterns in the eye movement data and one or more characteristics of one or more physiological states. The operations may further include outputting, by the machine-learning model, the physiological state of the user based on the identified associations. The operations may further include determining an intervention for the user based on the output physiological state. The operations may further include outputting the intervention.

In accordance with another embodiment, a system for automatically assessing a physiological state of a user may include an eye tracking device comprising a sensor, wherein the sensor is configured to capture eye movement of the user, the eye tracking device in electronic communication with a user device, one or more processors, and a memory including instructions, which, when executed by the one or more processors, cause the system to perform operations. The operations may include capturing, by the sensor, the eye movement as eye movement data. The operations may further include providing the eye movement data to a machine-learning model trained to identify associations between one or more patterns in the eye movement data and one or more characteristics of one or more physiological states. The operations may further include outputting, by the machine-learning model, the physiological state of the user based on the identified associations.

Additional objects and advantages of the disclosed aspects will be set forth in part in the description that follows, and in part will be apparent from the description, or may be learned by practice of the disclosed aspects. The objects and advantages of the disclosed aspects will be realized and attained by means of the elements and combinations discussed herein.

It is to be understood that both the foregoing general description and the following detailed description are exemplary and explanatory only and are not restrictive of the disclosed aspects.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a block diagram of an exemplary infrastructure implementing a physiological assessment system, according to one or more aspects of the present disclosure;

FIG. 2 is a data flow diagram of an exemplary method for generating a machine-learning model, according to one or more aspects of the present disclosure;

FIG. 3 is a flow diagram of an exemplary method for automatically assessing a physiological state of a user, according to one or more aspects of the present disclosure;

FIG. 4 depicts a flow diagram depicting a training of a machine-learning model, according to one or more embodiments; and

FIG. 5 depicts an example of a computing device, according to one or more embodiments.

### DETAILED DESCRIPTION

It is estimated that at least 80% of learning is derived from vision. It has long been understood that visual skills are important to evaluating a person's reading ability. Reading starts with vision and vision may be considered a dominant sense. After input from the eyes, the brain processes the visual content in order to make sense of what is seen. Higher-level brain processes may enable the reader to understand and comprehend text that is read. Both visual skills (e.g., using the eyes) and neural circuits (e.g., in the brain), together, may be considered imperative to effective and accurate understanding of text content.

Conventionally, it may be difficult to assess reading in an active manner, (e.g., while the reader is reading). This may make it difficult to identify relevant factors, determine appropriate interventions or corrections, and provide such in a timely manner (e.g., at a time that may impact learning and/or comprehension in real-time).

In one aspect of this disclosure, eye movement behavior may be evaluated using machine-learning or artificial intelligence (AI) techniques to identify various “states,” e.g., physical, mental, behavioral, or physiological characteristics or conditions. Examples of such states may include, but are not limited to, comprehension, confusion, fatigue, attention, and cognitive load, e.g., while engaged in the task of reading. In an exemplary embodiment, text may be identified in a reader's field of view, e.g., within a visual display viewed by the reader. Eye movement of the reader may be captured, e.g., using an eye tracker, camera, or any other suitable device. In some embodiments, a gaze of the reader's eyes is determined, e.g., to identify when the reader is actively engaged in reading. In some embodiments, other information about the environment is captured, e.g., motion, presence of distractions, objects, persons, etc. The reader's eye movement may be assessed against predetermined patterns and characteristics to determine one or more states. For example, the reader's eye movement may be evaluated using a machine-learning model that has been trained to coordinate various eye movements with various states. In response to determining a state for the reader, e.g., an undesirable state such as a lack of comprehension or a presence of confusion, one or more corrections or interventions may be applied or scheduled, e.g., during the reading or after at least a portion of the reading is complete.

In some instances, determination of a state may be binary, e.g., a state is present or is not present or is present to a threshold amount or within a threshold amount or certainty, or not. In some instances, determination of a state may indicate a degree, severity, or amount of the state. In various instances, a state may be determined at a particular point in time, for a portion of written material, for the written material as a whole, and etc. In some instances, a state may be determined over time, e.g., so that patterns or trends in an occurrence or severity of a state may be determined, or so that a future state may be predicted.

In various instances, a correction or intervention may be applied during or after interaction with written material by the reader. Such a correction or intervention may include an interaction or manipulation with the text of the written material, a cue or notification output to the reader or another person such as a teacher, proctor, supervisor, physician, or etc., a report or score regarding the reader's activity, or the like.

Various aspects of the present disclosure may also relate generally to determining one or more states of the reader, e.g., whether or to what extent the reader comprehends or is confused by written material as they engage with the material, as well as other activities such as selection and/or applying a correction or intervention. More particularly, a machine-learning model may be trained to coordinate eye movements to one or more states or degrees thereof, e.g., based on ground truth data relating to recorded eye movements with reader assessments.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as discussed herein.

In this disclosure, the term “based on” means “based at least in part on. ” The singular forms “a,” “an,” and “the” include plural referents unless the context dictates otherwise. The term “exemplary” is used in the sense of “example” rather than “ideal. ” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially,” “approximately,” “about,” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “reader,” “user,” participant,” student,” subject,” “patient,” etc., generally encompass any person involved in an activity that may include reading of written material from time to time. Terms like “provider,” “proctor,” “supervisor,” “employer,” reviewer,” or the like generally encompass an entity or person involved in providing written material, evaluating behavior or status of the reader, interacting with or benefiting from a reader's understanding of written material, or the like, as well as an agent or intermediary of such an entity or person. “Written material” generally encompasses any textual matter be it displayed electronically, printed, scribed, or otherwise.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as linear regression, logistic regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any combination of supervised or unsupervised techniques or ensemble techniques may also be used. Any suitable type of training may be used, e.g., stochastic, gradient boosted, random seeded, recursive, epoch or batch-based, etc.

While several of the examples herein involve certain types of machine-learning, it should be understood that techniques according to this disclosure may be adapted to any suitable type of machine-learning. Further, while some embodiments and/or examples pertain or refer to machine-learning, it should be understood that any suitable artificial intelligence technique may be used. It should also be understood that the examples above are illustrative only. The techniques and technologies of this disclosure may be adapted to any suitable activity.

While eye-tracking and various aspects relating to eye-tracking and target assessment results related to eye-tracking features are described in the present aspects as illustrative examples, the present aspects are not limited to such examples. For example, the present aspects can be implemented for or in combination with other types of feature tracking, such as facial recognition, body and/or kinesthetic movements (e.g., head movements), movements of objects (e.g., cars, planes, and the like), or in any other aspects where tracking features, movement, or progression may be measured and/or assessed. Further, the present systems and methods may be applied to methods and systems of evaluating comprehension, competence, confusion, etc. A software program (e.g., computer-readable instructions) and/or model (e.g., machine-learning model, artificial intelligence model, assessment model, or the like) may therefore become tuned, calibrated, or configured to evaluate any status or statuses.

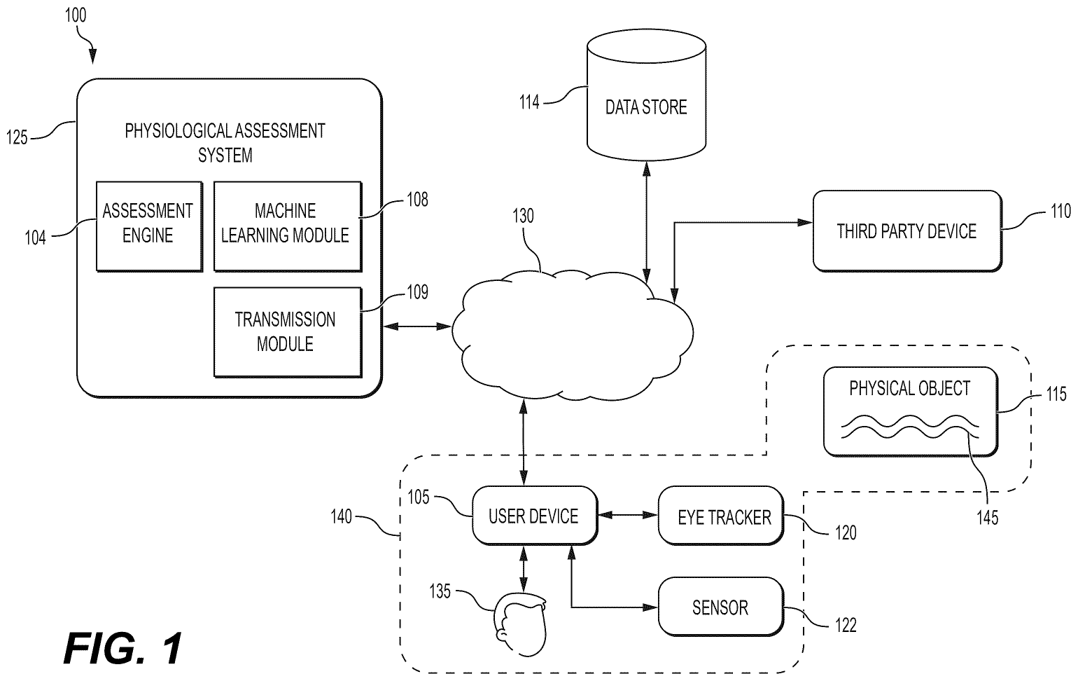

FIG. 1 depicts an exemplary infrastructure 100 implementing a physiological assessment system 125. The exemplary infrastructure 100 that may be utilized with techniques presented herein may include a user device 105, a third party device 110, a physical object 115, an eye tracker 120 (e.g., eye tracking device), a sensor 122, and a network 130 in communication with one or more of the other components of the infrastructure 100. One of more of the components, e.g., the user device 105, may be associated with a user 135 (e.g., a reader), and may be present, along with the user 135, in an environment 140.

As will be discussed in further detail below, one or more of the user device 105, third party device 110, physical object 115, or the like may depict, include, access, or display visual material or visual objects 145 (e.g., written material). The visual material 145 may be associated with a provider, or the like, which may also be associated, for example, with the physiological assessment system 125 or another element of the infrastructure 100.

The user device 105 may be configured to enable the user 135 to access and/or interact with other components of the infrastructure. For example, the user device 105 may include a computer system having a screen, display, or the like such as, for example, a desktop computer, a mobile device, a tablet, an augmented/virtual/extended reality (AR/VRXR) device (e.g., a headset, or glasses), an automobile, and/or heavy machinery, or the like (e.g., any suitable device having a display capable of outputting the visual material 145). In some embodiments, the user device 105 may include one or more electronic application(s), e.g., a program, plugin, browser extension, etc., installed on a memory of the user device 105. For instance, the user device 105 may include or access an assessment engine 104 configured to evaluate one or more state of the user 135. In some embodiments, the one or more electronic applications and/or components of infrastructure 100 may be accessed by the user device 105 over network 130 (e.g., such as in a cloud-based environment). For example, in some embodiments, the user device 105 may access data from the physiological assessment system 125 to obtain and output the visual material 145, evaluate the state(s) of the user 135, etc. In some embodiments, the electronic application(s) may be associated with one or more of the other components in the infrastructure 100.

The third party device 110 may include, for example, a display screen, a kiosk, or any other suitable device capable of including or displaying the visual material 145. The physical object 115 may include any suitable item may contain the visual material 145, e.g., a paper, a book, a blackboard, a billboard, a sign, etc. However, it should be understood that, in some embodiments, one or more objects in the environment 140 may not include the visual material 145, or the visual material 145 may not be visible in a field of view of the user 135, the eye tracker 120, the sensor 122, or the like.

The sensor 122 may include any suitable type of sensor operable to obtain information regarding the user 135 or the environment 140. For example, the sensor 122 may include a camera operable to determine a visual field of the user and/or one or more objects in the visual field of the user. In another example, the sensor 122 may include an accelerometer, e.g., to determine motion of the user device 105 or of the user 135, an audio sensor to detect audio signals in the environment 140, an infrared sensor, a heartbeat sensor, or the like.

In some embodiments, the eye tracker 120 may be integrated with the user device 105 or another component of the infrastructure 100. In some embodiments, the eye tracker 120 may be a separate component. In various embodiments, the eye tracker 120 may encompass any device that is capable of eye tracking, obtaining an eye-tracking signal, and/or gathering eye-tracking data and/or features, such that position as related to time (e.g., as a vector) may be captured with respect to eye-tracking, bodily movements (e.g., head movements), or the like. In examples, the eye tracker may include a camera, a medical device, an AR/VR/XR device, or the like.

The physiological assessment system 125 may be in electronic communication with a data store 114 (e.g., database). The data store 114 may include computer-readable memory such as a hard drive, flash drive, disk, etc. In some embodiments, the physiological assessment system 125 includes and/or interacts with an application programming interface for exchanging data to other systems, e.g., one or more of the other components of the infrastructure 100. The physiological assessment system may be implemented as a server. The physiological assessment system 125 and/or data store 114 may include and/or act as a repository or source for storing the visual material 145, a model or algorithm for determining a state of the user 135, tools for generating, training, or tuning such a model or algorithm, tools for determining or applying an intervention or correction, or the like.

Components of infrastructure 100 may electronically communicate over an electronic network 103. In various embodiments, electronic network 130 may be a wide area network (“WAN”), a local area network (“LAN”), personal area network (“PAN”), or the like. In some embodiments, electronic network 130 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

Although discussed as separate components, it should be understood that a component or portion of a component in the infrastructure 100 may, in some embodiments, be integrated with or incorporated into one or more other components. For example, components of physiological assessment system 125 may be embodied within an executable file (e.g., a configuration file) or a software program that is accessed, downloaded by, or otherwise integrated onto the user device 105, or the like.

In some embodiments, the components of the infrastructure 100 are associated with a common entity, e.g., a provider, or the like. The systems and devices of the infrastructure 100 may communicate in any arrangement. As will be discussed herein, systems and/or devices of the infrastructure 100 may communicate in order to one or more of display the visual material 145 to the user 135, track or capture eye movement of the user 135, determine a visual field of the user 135, identify aspects of the environment 140, detect reading activity of the user 135, evaluate the eye tracking of the user 135, e.g., to determine one or more status of the reader, determine one or more correction or intervention for the user 135, and provide a correction or intervention to the user 135 (e.g., via user device 105), among other activities. Further, as will be discussed in further detail below, the user device 105, physiological assessment system 125, or the like may one or more of (i) generate, store, train, communicate with, or use a machine-learning model and/or an artificial intelligence model configured to process eye-tracking data, e.g., to determine one or more state of the user 135.

As such, physiological assessment system 125 may include machine-learning module 108 that may include and/or implement the machine-learning model. In some embodiments, a system or device other than the physiological assessment system 125 may be used to generate and/or train the machine-learning model. For example, such a system may include instructions for generating the machine-learning model, the training data and ground truth, and/or instructions for training the machine-learning model. A resulting trained-machine-learning model may then be provided to the physiological assessment system 125.

As used herein, “machine-learning model” may be used interchangeably with one or more machine-learning and/or artificial intelligence models. Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn associations within eye-tracking data such that the trained machine-learning model is configured to output a target assessment result.

In various embodiments, the variables of a machine-learning model may be interrelated in any suitable arrangement in order to generate the output. For example, in some embodiments, the machine-learning model may include feature processing architecture that is configured to identify, isolate, and/or extract features in eye-tracking data. For example, the machine-learning model may include one or more convolutional neural network (“CNN”) configured to identify features in the eye-tracking data, and may include further architecture, e.g., a connected layer, neural network, etc., configured to determine a relationship between the identified features in order to determine an accurate target assessment result.

In some embodiments, the machine-learning model of the physiological assessment system 125 may include a Recurrent Neural Network (“RNN”). Generally, RNNs are a class of feed-forward neural networks that may be well adapted to processing a sequence of inputs. In some embodiments, the machine-learning model may include a Long Short Term Memory (“LSTM”) model and/or Sequence to Sequence (“Seq2Seq”) model. An LSTM model may be configured to generate an output from a sample that takes at least some previous samples and/or outputs into account. A Seq2Seq model may be configured to, for example, receive a sequence of eye-tracking data as input, and generate a target assessment result and/or an intervention.

As such, physiological assessment system 125 may include transmission module 109 that may transmit the intervention, output of the machine-learning model, or the like to the user device 105 for display.

In some embodiments, a plurality of machine-learning models may be used, e.g., in series, in parallel, and/or in conjunction with each other. For example, an image processing model, e.g., that includes a CNN or the like, may be used in conjunction with a camera or eye tracking device or the like to locate a user's gaze and/or to generate eye-tracking data, and an LSTM may be used to evaluate such data, e.g., to identify features in the data and/or generate evaluations based on such features.

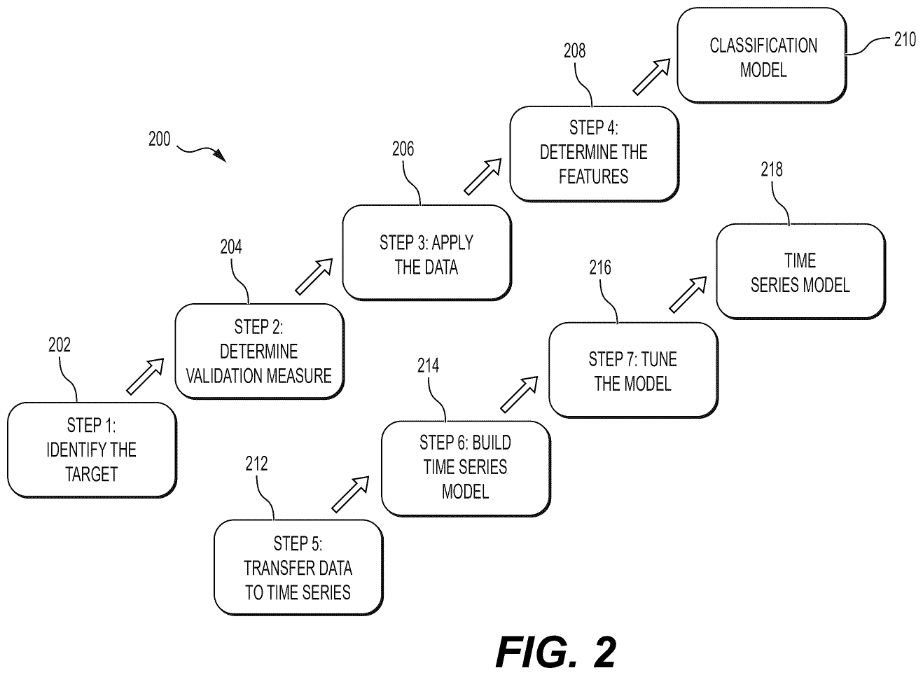

FIG. 2 depicts a data flow diagram of an exemplary method for generating a machine-learning model. In some embodiments, data may have been collected, e.g., over one or more assessments, studies, or the like, that includes ground truth data regarding how individuals comprehended reading material, along with eye-tracking data and/or environment data. In some instances, such data may not be of uniform content or format, e.g., across the different assessments, studies, or the like. Such data may thus be translated into a common schema and then used to develop a machine-learning model.

A classification model may be created. At step 202, the target may be identified. The target may be the variable or metric to predict. It may also be considered a dependent variable or the response variable. In examples, the target may include a cognitive load, a measure of fatigue, or a measure of stress. At step 204 a validation measure may be determined. The validation measure may be a ground truth or baseline standard. It may be a widely accepted measure of the target variable. For example, a NASA-TLX may be usable as a validation measure for cognitive load. The Visual Fatigue Survey (VFS) may be usable as a validation measure for fatigue. At step 206, the data may be applied. An assessment engine (e.g., such as assessment engine 104, as described with respect to FIG. 1) may analyze eye-tracking data to form solutions or outcomes. At step 208, features may be determined. Machine-learning methodology may be used to determine which eye-tracking features help identify the target state. The end result of steps 202-208 may be a classification model 210.

Next, a time series model may be created. At step 212, the data may be transferred to a time series. In examples, data is sequenced into segments to form time-series data. At step 214, the time series model may be built. Machine-learning and artificial intelligence (AI) may be used to build time series models, which may include forecasting, Long Short-Term Memory (LSTM) models, and the like. At step 216, the model may be tuned. Tuning parameters may be used to refine results. The end result of steps 212-216 may be a time series model.

In various implementations a transfer process may occur. An assessment engine (e.g., such as assessment engine 104, as described with respect to FIG. 1) may apply transfer learning to the pre-trained base models (e.g., the classification model and the time series model). In examples, transfer learning may be a type of supervised deep learning that involves transferring knowledge from one task to another. In this way, it may be possible to benefit from the general features and patterns learned by the pre-trained model to adapt them to related situations.

An exemplary transfer process may include decoupling algorithms from stimuli and testing models using various stimuli, testing using ground truth outcomes in new devices and firmware updates, deep learning with model parameter tuning with new trained layers, blind testing then un-blinding results against ground truth measures, stress testing models, and the like.

In examples, visual skills involved in reading may be used as the basis for determining metrics to use as ground truth for assessing one or more states. Visual skills for reading may include, for example visual counting (e.g., estimating a quantity of letters in a word, words in a sentence, and the like), voluntary eye movement control or saccades (e.g., capability to target the eye at the correct position of a word, on a page, and the like), fixation (e.g., capability to keep visual field stable), visual span (e.g., capability to extract and/or comprehend letters or words without refocusing), spatial attention (e.g., comprehension of orientation and order of letters or words), and/or visual acuity (e.g., identifying correct color, shape, identity, or the like of letters or words).

Skills such as the above visual skills may be evaluated via eye tracking, and may be associated with one or more eye movement behaviors, e.g., via the classification techniques discussed above. In this manner, eye movement data captured by an eye tracker may be correlated with various aspects of the activity of reading.

Supervised learning and validation may also be utilized. In various embodiments, one or more of the techniques above may include continued data collection using ground truth measures while tracking eye movements in different environments (e.g., cars, virtual reality headsets, space, augmented reality, laptops, and the like) while using different eye tracking solutions with different specifications (e.g., Hertz rates, precision measures, firmware updates, and the like). Ground truth assembled across different devices and environments may facilitate a robust model and may be usable to validate the model in such different environments and/or when operating on different devices.

Further, unsupervised learning and validation may be used. In some embodiments, a further process may take a supervised model (e.g., as discussed above), and test it against other eye-tracking data with no ground truth. Such testing may include a large quantity of data (e.g., 12 million datasets or the like), which may have been assembled over time and from a variety of assessments or the like.

Further, Few Shot Learning (FSL) and adaptive learning may be utilized: In some embodiments, the model may be configured to be retrained when thresholds are reached, such that new classifications may be generated as they appear in real time, e.g., along with new behavioral pathways. An LSTM model may retrain in real-time when certain thresholds are met, allowing the model to dynamically create new classifications as they emerge. This approach may enable the model to continuously learn and adapt to new, previously unclassified states, facilitating real-time dynamic user profiling.

Further aspects of the machine-learning model and/or how it may be utilized to process eye-tracking are discussed in further detail in the methods below. Additionally, further aspects of the determination of one or more physiological states of a user as well as corrections or interventions for the user are discussed in further detail in the methods below. In the following methods, various acts may be described as performed or executed by a component from FIG. 1, such as the user device 105, the eye tracker 120, etc. However, it should be understood that in various embodiments, various components of the infrastructure 100 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

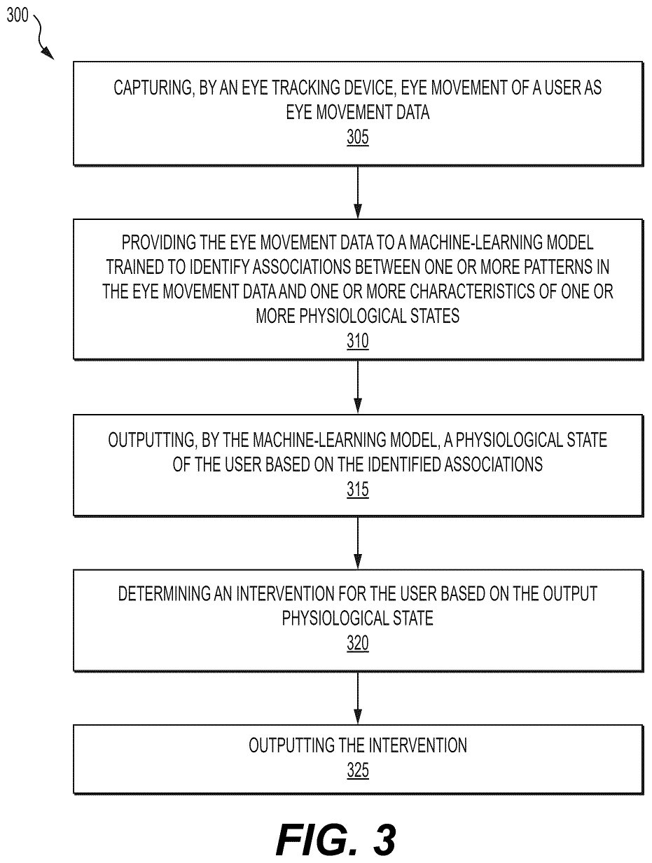

FIG. 3 is a flow diagram depicting an exemplary method for automatically assessing a physiological state of a user. A user may be located at an environment (e.g., such as user 135 in environment 140, as described with respect to FIG. 1). The user may be associated with, for example, a user device (e.g., user device 105, as described with respect to FIG. 1), an eye tracker (e.g., eye tracker 120, as described with respect to FIG. 1), or the like. One or more physical objects in the environment may include visual objects (e.g., such as visual material 145, as described with respect to FIG. 1), such as readying material. In particular embodiments, and prior to step 305, which will be described below, it may be determined that the user is engaging with visual material. The engaging may include eye movement. In examples, the detection of eye movement of the user may indicate that the user in engaging with the visual material, visual objects, and/or physical object(s) in the environment around the user. In some examples, a visual display including the visual material may be displayed on a display of the user device. The visual material may be configured to elicit a response from the user that includes eye movement. In one example, the user device, a third party device, or the like may include a screen configured to display the visual objects and/or material, such as text. In another example, a paper, book, sign, whiteboard, blackboard, or another physical object in an environment around the user, or the like may include the visual material. In various embodiments, instead of the user engaging with visual objects and/or visual material, the user may be positioned within a range of the eye tracker (e.g., eye tracking device). The eye tracker may therefore be configured to capture eye movements of the user (e.g., passively) without the user being actively engaged with visual objects and/or visual material. For example, while some physiological states may be associated with or visible during a user's engagement with visual object or visual material (e.g., reading comprehension attention, etc.), some physiological states may be evident via evaluation of eye tracking whether or not a user is engaging with a visual object or visual material (e.g., fatigue, stress, executive function, etc.).

In various embodiments, an assessment engine (e.g., such as assessment engine 104, as depicted in FIG. 1) may identify the presence of visual objects in the environment. In an example, the assessment engine may operatively communicate with an eye tracking device or sensor (e.g., a camera or the like) to capture eye movements. At step 305, the eye movement may be captured by the eye tracking device (and/or sensor) as eye movement data. In some instances, the eye tracker and/or sensor may be oriented to have a view that at least partially overlaps with a field of view of the user. For instance, the sensor may include a front camera on a phone, a forward facing camera on an AR device, or the like.

In some embodiments, a text and/or object detection algorithm may be configured to process data from the sensor to identify any viewed text or visual objects/material, e.g., in real or near-real time. In another example, the assessment engine may operatively communicate with the user device having the screen displaying the text. For example, the assessment engine may have access to or awareness of the display of the user device, the third party device, or the like. In some embodiments, a text detection algorithm may be applied to screen data from such a user device. Any suitable text detection algorithm or model may be used, e.g., various forms of machine-learning (ML) or Artificial Intelligence (AI) or any other method for real time identification of information in an environment. In some embodiments, the eye tracker may be configured to capture eye movements of the user (e.g., passively) without the user being actively engaged with visual objects and/or visual material. The eye movements may therefore be provided to the assessment engine in such embodiments.

In some embodiments, the identification of text or visual objects in the environment may be continuous. In some embodiments, the identification is performed responsive to a stimulus or instruction. For instance, a program or schedule may indicate a start of a reading period, and the assessment engine may receive eye-tracking data from an eye tracker (e.g., such as eye tracker 120, as described with respect to FIG. 1) indicative of active reading, or a user instruction (e.g., from the reader user or the provider) which may initiate the identifying process, or the like.

The eye tracker may capture and/or record eye-tracking data. In various embodiments, eye-tracking data may include, for example, one or more x, y, z coordinates of eye location and/or gaze direction along with time stamps. The gaze of the user's eyes may therefore be captured. The captured gaze may identify that the user is actively engaging with the visual display. The captured gaze may also provide eye tracking data that, when provided to a machine-learning model for assessment or analysis, such as those machine-learning models described herein, may indicate a physiological state whether or not a user is engaging with a visual object or visual material (e.g., for such physiological states such as fatigue, stress, executive function, etc.). The eye tracker and/or the assessment engine may also capture, for example, one or more look-zones within the field of view of the user, e.g., via the eye tracker, the sensor, and/or a component in communication with a device outputting a display. A look zone may include, for example, a location of a paragraph start, a paragraph end, individual words (e.g., predetermined or identified via a context model), sentences (e.g., key or topic sentences that are predetermined or identified via the context model), as well as other stimuli or spaces (e.g., of a room) that may be within the field of view. For example, one or more portions of the display output by a screen, e.g., a portion separate from a portion that includes text, an object or person in the background, etc., may act as a distraction during reading or engagement with the visual objects/material. In further examples, a user may not be engaged with any such visual objects and one or more look-zones with a field of view of the user may be captured by the eye tracker and/or sensor. It should be understood that the data captured may relate to content of the visual objects (e.g., such as visual material 145, as described with respect to FIG. 1) and/or unrelated material in the environment (e.g., such as environment 140, as described with respect to FIG. 1), such as factors or objects that may have an impact on reading activity or engagement with the visual display of the visual objects/material, or a user's field of view. Therefore, one or more environmental factors in the environment around the user device may be captured by the eye tracker and/or sensor in electronic communication with the user device.

Such eye-tracking data and other captured data may be provided to the assessment engine, which may employ a trained machine-learning model to determine one or more states of the user, e.g., in real or near-real time. Therefore, at step 310, the eye movement data may be provided to the machine-learning model, the machine-learning model trained to identify associations between one or more patterns in the eye movement data and one or more characteristics of one or more physiological states. Further aspects regarding the training and development of such a model are discussed in further detail below. In various implementations, the one or more environmental factors may be provided to the machine-learning model along with the eye movement data and the one or more characteristics of the one or more physiological states. The machine-learning model may then adjust the output physiological state of the user based on the identified associations between the environmental factors, the eye movement data, and the one or more characteristics of the one or more physiological states.

Examples of physiological states that may be determined by the machine-learning model may include, but are not limited to, a physical, mental, behavioral, or physiological condition, comprehension (e.g., reading), confusion, fatigue, cognitive load, attention, interest, boredom, and the like. At step 315, the machine-learning model may output a physiological state of the user based on the identified associations. In examples, the physiological state may indicate a performance of the user's engagement with visual material (e.g., with respect to a physical, mental, behavioral, or physiological condition, reading comprehension, confusion, fatigue, cognitive load, attention, interest, boredom, and the like). In examples, the physiological state may indicate a physiological condition of the user whether or not the user is engaging with visual objects. Information regarding the determined state(s) of the user may be stored (e.g., to a data store via the assessment engine) and/or transmitted or reported to any suitable device (e.g., such as by transmission module 109, as described with respect to FIG. 1). For example, a provider, e.g., a teacher, may be provided with a live feed describing whether the reading of the visual objects (e.g., text) is being comprehended, or that the user is confused, fatigued, bored, or the like. In other examples, the physiological state of the user may be provided to a physician or other professional (e.g., occupational therapist, law enforcement officer, or the like). In various embodiments, the machine-learning model may also output a value associated with the physiological state of the user based on the identified associations. The value may indicate a degree of severity of the physiological state. For example, a degree of fatigue, a degree of the lack of comprehension, and the like.

In some embodiments, the machine-learning model may record or track one or more states over time. In some embodiments, the machine-learning model may be configured to determine patterns or trends in one or more states. For example, the machine-learning model may determine that the reader's confusion is increasing, and thus may be likely to increase beyond a predetermined threshold. In other examples, the machine-learning model may determine that a user's determined physiological state is worsening or improving (e.g., becoming more fatigued, or visa versa). In some embodiments, one physiological state may be used to determine a trend, pattern, or prediction in another. For example, an increasing fatigue measurement may be used to predict a decrease in comprehension or attention at a future point in time. Therefore, the eye movement may be captured by the eye tracking device in electronic communication with the user device as eye movement data over a period of time. The eye movement data captured over the period of time may be provided to the machine-learning model trained to identify associations between one or more patterns in the eye movement data captured over the period of time and the one or more characteristics of the one or more physiological states. The machine-learning model may then adjusting the output physiological state of the user based on the identified associations over the period of time (e.g., based on the degree, increase, decrease, of the state, or the like).

In some embodiments, eye-tracking data may be used alongside or instead of physiological state data to predict future states for the user. In some embodiments, other information about the environment, e.g., the other data captured alongside the eye-tracking data, may be used to make predictions regarding one or more states. For example, the presence of a loud conversation, a person moving into frame, or the like, may be used to predict a drop in attention, or the like. Various embodiments may combine one or more of the foregoing. In some embodiments, eye-tracking data, state data, and/or environment data may be provided to a prediction algorithm or model, e.g., a predictive machine-learning model, which is trained to find associations in the data and output predictions about a future physiological state of the user. In an example, an LSTM model may be used to make a prediction regarding one or more states.

In some embodiments, the assessment engine may use the output state data and/or other data to generate a post-reading or post-assessment report. Such a report may include, for example, a profile for the user including various characteristics of the user and/or their interaction with the visual display of the visual objects/material, overall statistics of one or more states of the user, a breakdown of one or more states over time or correlated against words or portions of the text (e.g., visual objects) as they were read, and/or one or more scores for the user, e.g., based on a comparative assessment of the one or more states against a baseline and/or one or more other individuals, or the like.

At step 320, an intervention for the user based on the output physiological state may be determined. In some embodiments, the assessment engine may determine and/or generate one or more corrections or interventions for the user. In some embodiments, the assessment engine may utilize a display of the user device (e.g., outputting the written material and/or the display of an AR device) to overlay content on top of the visual objects/material or within a visible area of the environment (e.g., such as environment 140, as described with respect to FIG. 1). At step 325, the intervention may be output (e.g., by physiological assessment system 125 via transmission module 109, or user device 105, as described with respect to FIG. 1, or the like). In examples, the intervention may be displayed on the display of the user device. Examples of corrections or interventions may include, for example, displaying a text notification, highlighting a word or sentence, e.g., a keyword, important concept, portion or the like, increasing a size of the text, increasing a line spacing of the text, adding cues to identify text portions such as line numbers, paragraph numbers, or the like, changing text or background color, adjusting a zoom or focus of the camera, displaying an auditory notification, causing the user device to output a haptic notification, displaying an augmented reality overlay with one or more other visual objects/material, and the like, or combinations thereof. A notification may include, for example, an indication of one or more states of the user, a reference to a portion of the visual objects/material, a call to action, e.g., to return to reading or to re-read a particular portion, and the like. Corrections or interventions may be generated and or applied in real or near-real time while the user is reading or engaging with the visual objects/material, after the user has finished reading or engaging with a portion, and/or after the user has concluded reading or engaging with the visual objects/material.

To train a machine-learning model to determine one or more physiological states of the user, data may be leveraged that includes both eye-tracking data and data indicative of one or more physiological states. In an example, a person may be provided with a passage to read or a visual object with which to interact. As the person reads, their eye movements may be tracked. After the reading, the person may be provided with a comprehension exercise related to specific content in the passage, e.g., as a whole or to portions thereof. The answers to the exercise may be used to identify whether and/or to what extent the person comprehended the passage or specific portions thereof. Training of a machine-learning model may be used to coordinate particular eye movement behaviors to comprehension or lack thereof. In another example, particular eye movements, e.g., regressions (the person re-reading a portion of the passage), or the like may be correlated to a state of confusion. Eye movement away from the passage may correlate with a state of inattention, and so on. In further embodiments, the machine-learning model may be trained using eye-tracking data and data known to indicate one or more physiological states (e.g., a ground truth). Such data known to indicate one or more physiological states may include data known to indicate fatigue, cognitive load, impairment, and the like.

In various embodiments, any physiological state that may be determined via any type of assessment or exercise may be correlated with eye movements in order to associate particular eye movement behavior with an indication of or change in the state. For example, while a person is reading a passage, any suitable sensor may capture data regarding a characteristic of the person, and such characteristics may be evaluated to determine a state. That state may then be correlated with the person's eye movements during the reading. As a result, once the model is trained, the model may be used to assess the state of a person, e.g., without necessarily using the sensor or sensors used to capture the characteristic, but instead relying on the correlated eye movement behaviors.

Any suitable technique for developing or training such a model may be used. In various implementations, a classification model may trained to identify features that detect a particular target (e.g., comprehension, confusion, fatigue, cognitive overload, and the like). In an exemplary embodiment, the identified features may be utilized as input into the trained assessment model. One or more gathered or simulated sets of eye-tracking features data may be provided to one or more target assessment algorithms as one or more sets of training data. The one or more target assessment algorithms may determine associations (e.g., using the features identified by the classification model) between the one or more gathered or simulated sets of eye-tracking features data and one or more target assessment results (e.g., fatigue, cognitive overload, and the like).

One or more of a layer, a weight, a synapse, or a node of the first assessment model may be modified based on the determined associations between the one or more gathered or simulated sets of eye-tracking features data and the one or more target assessment results. As a result, an assessment model may be output (e.g., as a trained machine-learning model). In examples, the output may be a predicted or determined target. The assessment model may therefore be trained to determine the target assessment result based on the set of eye-tracking features data and output a first assessment result based on the set of eye-tracking features data and the modified one or more of the layer, the weight, the synapse, or the node of the first assessment model. In various implementations, the first assessment model may be retrained using one or more first assessment results and the one or more gathered or simulated sets of eye-tracking features data.

In another example, a target state and associated data are identified to determine eye-tracking features. In a particular example, if the target is fatigue, then the associated data may include eye-related features that signify fatigue. In the example, the eye-tracking features may then be determined to include those eye-related features that may be observed and/or measured using eye-tracking (e.g., using eye tracker 120). For instance, one or more machine-learning classification techniques may compare eye-tracking data against predetermined or ground truth fatigue data in order to determine features in the eye-tracking data that are indicative of the fatigue data. An assessment model trained using the identified target state, associated data, and determined eye-tracking features may be output. In various examples, the assessment model may therefore be trained to identify the target state based on gathered and/or simulated eye-tracking features. The output data from the first assessment model may be input into a time series, which may be input into a second model in order to train the second model to generate predictions regarding the identified target. In an example, the second model may include a long short-term (LSTM) model, a recurrent model, a sequence-based model, a transformer, or any other suitable model capable of learning and/or predicting events, trends, patterns, or the like. In an example, the time series data regarding the determined features may be evaluated against subsequent events in the time series, so that the second model is trained to make predictions regarding the target based on preceding features in time series data. The output of the second model may then be compared to an output of the first assessment model (e.g., a comparison of a prediction to occur at a later time Y based on features detected at earlier time X using the second model against a classification of features detected using the first model at time Y) to validate the output of the second model.

In various embodiments, output data of the calibrated assessment model may be input into a third model. The output of the third model may be compared to an output of the calibrated assessment model to validate the output of the third model. In various embodiments, the third model may be configured to identify variations in the identified target. For example, fatigue, rather than just being a binary indicator of true or false, may have gradations or levels, e.g., low, medium or high. The third model may be trained, e.g., based on gradation data, to predict a future time in the time series at which the gradation or level of the target is likely to change, e.g., low fatigue changes to medium, etc. A low-medium-high assessment model may be output, having been trained using the comparison. In various embodiments, the low-medium-high assessment model may be trained to determine associations between one or more gathered and/or simulated sets of eye-tracking features and one or more target assessment results and output one or more predictions of a target. In an example where the target state is fatigue, the prediction of the target state may include a determination that the user is not fatigued currently, but that the user will be fatigued within a determined period of time (e.g., based on continuing a current level of activity, or the like). Such predictions may be used to enact a correction or intervention, e.g., before the undesired state occurs or worsens.

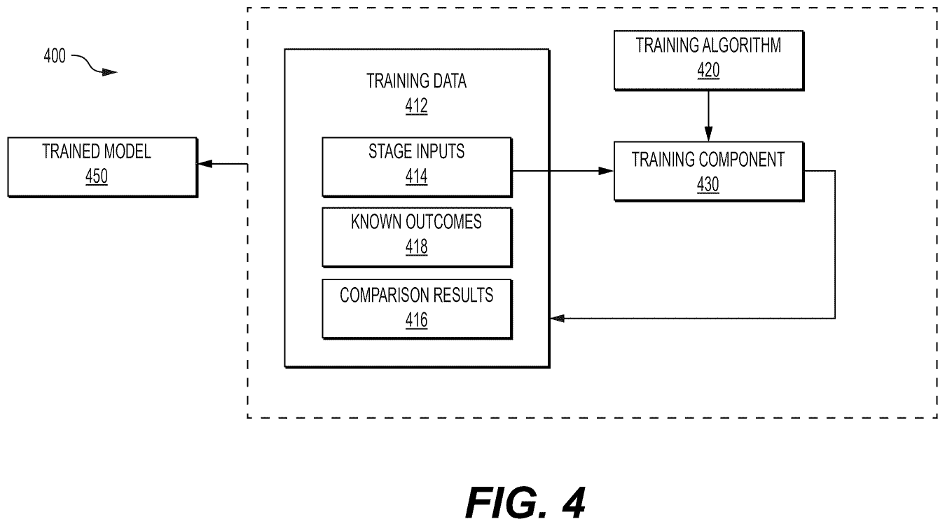

FIG. 4 depicts a flow diagram for training a machine-learning model. As shown in flow diagram 400 of FIG. 4, training data 412 may include one or more of stage inputs 414 and known outcomes 418 related to a machine-learning model to be trained. The stage inputs 414 may be from any applicable source including a component or set shown in the figures provided herein. The known outcomes 418 may be included for machine-learning models generated based on supervised or semi-supervised training. An unsupervised machine-learning model might not be trained using known outcomes 418. Known outcomes 418 may include known or desired outputs for future inputs similar to or in the same category as stage inputs 414 that do not have corresponding known outputs.

The training data 412 and a training algorithm 420 may be provided to a training component 430 that may apply the training data 412 to the training algorithm 420 to generate a trained machine-learning model 450. According to an implementation, the training component 430 may be provided comparison results 416 that compare a previous output of the corresponding machine-learning model to apply the previous result to re-train the machine-learning model. The comparison results 416 may be used by the training component 430 to update the corresponding machine-learning model. The training algorithm 420 may utilize machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of the flow diagram 400 may be a trained machine-learning model 450.

A machine-learning model disclosed herein may be trained by adjusting one or more weights, layers, and/or biases during a training phase. During the training phase, historical or simulated data may be provided as inputs to the model. The model may adjust one or more of its weights, layers, and/or biases based on such historical or simulated information. The adjusted weights, layers, and/or biases may be configured in a production version of the machine-learning model (e.g., a trained model) based on the training. Once trained, the machine-learning model may output machine-learning model outputs in accordance with the subject matter disclosed herein. According to an implementation, one or more machine-learning models disclosed herein may continuously update based on feedback associated with use or implementation of the machine-learning model outputs.

It should be understood that aspects in this disclosure are exemplary only, and that other aspects may include various combinations of features from other aspects, as well as additional or fewer features. For example, the present aspects can be implemented for various types of fields, such as in any scenario related to optimizing data, detecting anomalies, generating alerts, predicting outcomes, and the like.

In general, any process or operation discussed in this disclosure that is understood to be computer-implementable, such as the processes illustrated in the flowcharts disclosed herein, may be performed by one or more processors of a computer system, such as any of the systems or devices in the exemplary environments disclosed herein, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices disclosed herein. One or more processors of a computer system may be included in a single computing device or distributed among a plurality of computing devices. A memory of the computer system may include the respective memory of each computing device of the plurality of computing devices.

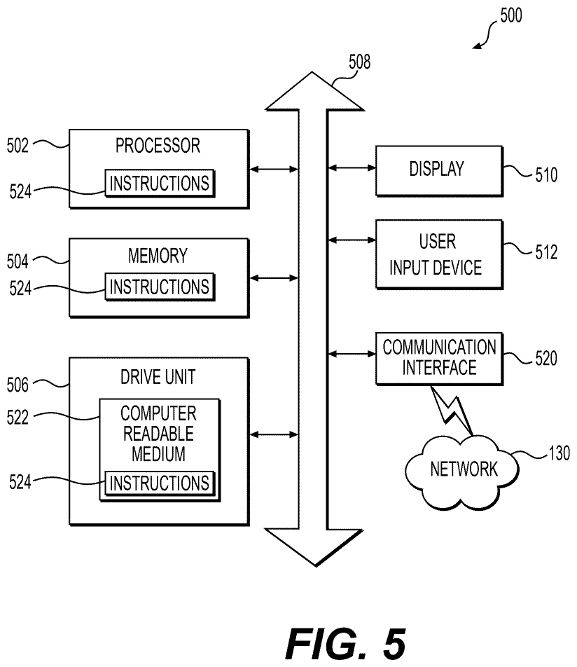

FIG. 5 is a simplified functional block diagram of a computer 500 that may be configured as a device for executing the methods disclosed here, according to exemplary aspects of the present disclosure. For example, the computer 500 may be configured as a system according to exemplary aspects of this disclosure. In various aspects, any of the systems herein may be a computer 500 including, for example, a data communication interface 520 for packet data communication. The computer 500 also may include a central processing unit (“CPU”) 502, in the form of one or more processors, for executing program instructions. The computer 500 may include an internal communication bus 508, and a storage unit 506 (such as ROM, HDD, SDD, etc.) that may store data on a computer readable medium 522, although the computer 500 may receive programming and data via network communications.

The computer 500 may also have a memory 504 (such as RAM) storing instructions 524 for executing techniques presented herein, for example the methods described with respect to FIG. 3, although the instructions 524 may be stored temporarily or permanently within other modules of computer 500 (e.g., processor 502 and/or computer readable medium 522). The computer 500 also may include input and output ports 512 and/or a display 510 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

These and other embodiments of the systems and methods may be used as would be recognized by those skilled in the art. The above descriptions of various systems and methods are intended to illustrate specific examples and describe certain ways of making and using the systems disclosed and described here. These descriptions are neither intended to be nor should be taken as an exhaustive list of the possible ways in which these systems can be made and used. A number of modifications, including substitutions of systems between or among examples and variations among combinations can be made. Those modifications and variations should be apparent to those of ordinary skill in this area after having read this disclosure.

The systems, apparatuses, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. In this disclosure, any identification of specific techniques, arrangements, etc., are either related to a specific example presented or are merely a general description of such a technique, arrangement, etc. Identifications of specific details or examples are not intended to be, and should not be, construed as mandatory or limiting unless specifically designated as such. Any failure to specifically describe a combination or sub-combination of components should not be understood as an indication that any combination or sub-combination is not possible. It will be appreciated that modifications to disclosed and described examples, arrangements, configurations, components, elements, apparatuses, devices, systems, methods, etc., can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures, or characteristics may be combined in any suitable manner in one or more embodiments.

Throughout this disclosure, references to components or modules generally refer to items that logically can be grouped together to perform a function or group of related functions. Like reference numerals are generally intended to refer to the same or similar components. Components and modules can be implemented in software, hardware, or a combination of software and hardware. The term “software” is used expansively to include not only executable code, for example machine-executable or machine-interpretable instructions, but also data structures, data stores and computing instructions stored in any suitable electronic format, including firmware, and embedded software. The terms “information” and “data” are used expansively and includes a wide variety of electronic information, including executable code; content such as text, video data, and audio data, among others; and various codes or flags. The terms “information,” “data,” and “content” are sometimes used interchangeably when permitted by context. It should be noted that although for clarity and to aid in understanding some examples discussed herein might describe specific features or functions as part of a specific component or module, or as occurring at a specific layer of a computing device (for example, a hardware layer, operating system layer, or application layer), those features or functions may be implemented as part of a different component or module or operated at a different layer of a communication protocol stack. Those of ordinary skill in the art will recognize that the systems, apparatuses, devices, and methods described herein can be applied to, or easily modified for use with, other types of equipment, can use other arrangements of computing systems such as client-server distributed systems, and can use other protocols, or operate at other layers in communication protocol stacks, than are described.

It is intended that the specification and examples be considered as exemplary only, with a true scope and spirit of the invention being indicated by the following claims.

What is claimed is:

1. A computer-implemented method for automatically assessing a physiological state of a user, the method comprising:
capturing, by an eye tracking device, eye movement of the user as eye movement data;
providing, by one or more processors, the eye movement data to a machine-learning model trained to identify associations between one or more patterns in the eye movement data and one or more characteristics of one or more physiological states;
outputting, by the machine-learning model, the physiological state of the user based on the identified associations;
determining, by the one or more processors, an intervention for the user based on the output physiological state; and
outputting, by the one or more processors, the intervention.

2. The computer-implemented method of claim 1, wherein the physiological state indicates a performance of an engagement of the user with visual material.

3. The computer-implemented method of claim 1, wherein the intervention is delivered via a user device and is one or more of an audio cue, visual display, notification, and haptic feedback.

4. The computer-implemented method of claim 1, further comprising:
capturing, by the eye tracking device, a gaze of the user's eyes.

5. The computer-implemented method of claim 1, further comprising:
capturing, by a sensor in electronic communication with a user device, one or more environmental factors in an environment around the user device;
providing, by the one or more processors, the one or more environmental factors to the machine-learning model trained to identify associations between the one or more patterns in the eye movement data, the one or more characteristics of the one or more physiological states, and the one or more environmental factors; and
adjusting, by the machine-learning model, the output physiological state of the user based on the identified associations.

6. The computer-implemented method of claim 1, further comprising:
outputting, by the machine-learning model, a value associated with the physiological state of the user based on the identified associations, wherein the value indicates a degree of severity of the physiological state.

7. The computer-implemented method of claim 1, further comprising:
capturing, by the eye tracking device, the eye movement as eye movement data over a period of time;
providing, by the one or more processors, the eye movement data captured over the period of time to the machine-learning model trained to identify associations between one or more patterns in the eye movement data captured over the period of time and the one or more characteristics of the one or more physiological states; and
adjusting, by the machine-learning model, the output physiological state of the user based on the identified associations over the period of time.

8. A system for automatically assessing a physiological state of a user, the system comprising:
an eye tracking device configured to capture eye movement of the user, the eye tracking device in electronic communication with a user device;
one or more processors; and
a memory including instructions, which, when executed by the one or more processors, cause the system to perform operations including:
capturing, by the eye tracking device in electronic communication with the user device, the eye movement as eye movement data;
providing, by the one or more processors, the eye movement data to a machine-learning model trained to identify associations between one or more patterns in the eye movement data and one or more characteristics of one or more physiological states;
outputting, by the machine-learning model, the physiological state of the user based on the identified associations;
determining, by the one or more processors, an intervention for the user based on the output physiological state; and
outputting, by the one or more processors, the intervention.

9. The system of claim 8, wherein the physiological state indicates a performance of an engagement of the user with visual material.

10. The system of claim 8, wherein the physiological state is a measure of one or more of comprehension, confusion, fatigue, attention, and cognitive load.

11. The system of claim 8, the operations further comprising:
capturing, by the eye tracking device in electronic communication with the user device, a gaze of the user's eyes.

12. The system of claim 8, further comprising:
a sensor in electronic communication with the user device, wherein the operations further comprise:
capturing, by the sensor, one or more environmental factors in an environment around the user device;
providing, by the one or more processors, the one or more environmental factors to the machine-learning model trained to identify associations between the one or more patterns in the eye movement data, the one or more characteristics of the one or more physiological states, and the one or more environmental factors; and
adjusting, by the machine-learning model, the output physiological state of the user based on the identified associations.

13. The system of claim 8, the operations further comprising:
outputting, by the machine-learning model, a value associated with the physiological state of the user based on the identified associations, wherein the value indicates a degree of severity of the physiological state.

14. The system of claim 8, the operations further comprising:
capturing, by the eye tracking device in electronic communication with the user device, the eye movement as eye movement data over a period of time;
providing, by the one or more processors, the eye movement data captured over the period of time to the machine-learning model trained to identify associations between one or more patterns in the eye movement data captured over the period of time and the one or more characteristics of the one or more physiological states; and
adjusting, by the machine-learning model, the output physiological state of the user based on the identified associations over the period of time.

15. A system for automatically assessing a physiological state of a user, the system comprising:
an eye tracking device comprising a sensor, wherein the sensor is configured to capture eye movement of the user, the eye tracking device in electronic communication with a user device;
one or more processors; and
a memory including instructions, which, when executed by the one or more processors, cause the system to perform operations including:
capturing, by the sensor, the eye movement as eye movement data;
providing, by the one or more processors, the eye movement data to a machine-learning model trained to identify associations between one or more patterns in the eye movement data and one or more characteristics of one or more physiological states; and
outputting, by the machine-learning model, the physiological state of the user based on the identified associations.

16. The system of claim 15, wherein the physiological state indicates a performance of an engagement of the user with visual material.

17. The system of claim 15, wherein the physiological state is a measure of one or more of comprehension, confusion, fatigue, attention, and cognitive load.

18. The system of claim 15, the operations further comprising:
capturing, by the sensor, a gaze of the user's eyes.

19. The system of claim 15, the operations further comprising:
outputting, by the machine-learning model, a value associated with the physiological state of the user based on the identified associations, wherein the value indicates a degree of severity of the physiological state.

20. The system of claim 15, the operations further comprising:
capturing, by the sensor, the eye movement as eye movement data over a period of time;
providing, by the one or more processors, the eye movement data captured over the period of time to the machine-learning model trained to identify associations between one or more patterns in the eye movement data captured over the period of time and the one or more characteristics of the one or more physiological states; and
adjusting, by the machine-learning model, the output physiological state of the user based on the identified associations over the period of time.
